(19) United States
(12) Patent Application Publication
(10) Pub. No.: US 2002/0183857 A1
(43) Pub. Date: Dec. 5, 2002

(54) Vascular tissue composition

(76) Inventor: Jun Yang, Dove Canyon, CA (US)

Correspondence Address: Raymond Sun, Law Offices of Raymond Sun, 12420 Woodhall Way, Tustin, CA, 92782, US

(21) Appl. No.: 10/195,276

(22) Filed: Jul. 15, 2002

Related U.S. Application Data
(62) Division of application No. 09/755,818, filed on Jan. 5, 2001, now abandoned.

(51) Int. Cl. A61F 2/02

(52) U.S. Cl. 623/23.72; 623/13.17; 623/14.12; 623/916; 435/378

(57) ABSTRACT

A tissue composition includes the subendothelial layer, the elastica interna, and at least a portion of the tunica media of a blood vessel harvested from a mammal, with the endothelial cells removed from the blood vessel. The tissue composition can also include a portion of the tunica adventitia of a blood vessel harvested from a mammal. The tissue composition can be formed into a graft, a patch, a connective tissue for surgical repair, an orthopedic graft, and a substrate for cell growth, among other applications. The tissue composition can also be fluidized, or made into powdered form.

### BACKGROUND OF THE INVENTION

1. Field of the Invention

The present invention relates to a novel tissue composition exhibiting low immunogenicity and thrombogenicity, high strength, variable absorbability, and capable of promoting endogenous tissue growth, and in particular, to vascular tissue compositions that comprise the subendothelial layer, the elastica interna, and either a portion of the tunica media or the tunica media and a portion of the tunica adventitia.

2. Description of the Prior Art

Both synthetic and autogenous materials have been used for grafting. Among synthetics, ePTFE and Dacron have been widely used as vascular grafting materials. Unfortunately, they are susceptible to neointimal hyperplasia, early and late thrombosis, and chronic inflammation around the grafts. In human clinicals, at the luminal surface, endothelial cells have been seen only within a few millimeters on the synthetic graft from the anastomosis, while on the outside of the synthetic graft, there is a severe fibrous encapsulation around the graft. For non-vascular applications, synthetic grafting material usually does not promote early host cell attachment and growth.

Among autogenous and allogenous materials, the saphenous vein, the human umbilical vein, the inverted small intestine, and the radial artery have all been used, but they have also experienced significant shortcomings. The saphenous vein may be of an inappropriate size for certain procedures or may be unavailable because of damage by disease. In addition, the saphenous vein may have unacceptable varicosities and suffers from accelerated atherogenisis following arteriolization. Both the umbilical grafts and the inverted small intestine grafts are plagued by early thrombosis and late aneurysm formation. Finally, the radial artery is of limited utility because it is difficult to harvest and may deteriorate after graft implantation.

More recently, researchers have reported preparation of tissue graft utilizing small intestine submucosa (SIS). The SIS has many drawbacks, such as relative inconsistency of tissue composition (e.g., cell populations, protein, lipid composition), the possibility of contamination from food and bacteria, the presence of a high level of products from the local immune response (e.g., lymphocytes, macrophages, cytokines) within the SIS, and residual local bacteria within the SIS.

In addition to the above, researchers in the surgical arts have been working for many years to develop new techniques and materials for use as grafts to replace or repair damaged or diseased tissue structures, particularly bones and connective tissues such as ligaments and tendons, and to hasten fracture healing. For example, it is very common today for an orthopedic surgeon to harvest a patellar tendon of autogenous or allogenous origin for use as a replacement for a torn cruciate ligament. The surgical methods for such techniques are well known. Further it has become common for surgeons to use implantable prostheses formed from plastic, metal and/or ceramic material for reconstruction or replacement of physiological structures. Yet despite their wide use, surgically implanted prostheses present many attendant risks to the patient. For example, they do not usually promote complete healing of the damaged natural tissue structure. On the other hand, the presence of an implanted prosthesis may promote chronic inflammation (scar tissue) as a result of the host response against the foreign material. As a result, surgeons are in need of a non-immunogenic, high tensile strength graft material which can be used for surgical repair of bones, tendons, ligaments and other functional tissue structures.

In addition, researchers have been attempting to develop satisfactory polymer or plastic materials to serve as ligament or tendon replacements, or to enhance weakened structures for other connective tissues, such as those involved in hernias and. It has been found that it is difficult to provide a tough, durable plastic material which is suitable for long-term connective tissue replacement. In addition, plastic materials can become infected and difficulties in treating such infections often lead to graft failure.

Diaphyseal bone loss is another medical condition that has previously been treated with grafting. Diaphyseal bone loss results from high-energy trauma, infection, or resection performed for skeletal neoplasia. The numerous treatment modalities attest to the difficulty in obtaining reliable and satisfactory reconstitution of skeletal long bone defects. Methods for treating diaphyseal bone defects include: autologous cancellous or cortical bone grafts, autologous vascularized bone transplants and grafts, xenogenic bone grafts, allogenic bone grafts, hydroxyapatite and/or tricalcium phosphate ceramics implants, coral, natural polymers like collagen, synthetic polymers like polyethylene and polyglycolide, osteoinductive proteins such as bone morphogenetic protein, and bone transport techniques. All these methods have failed to produce predictable reconstruction of cortico-cancellous bone loss from diaphyseal segment of the human skeleton.

In yet another application, proposals have been made for the application of membranes containing resorbable or degradable polymeric material for bone regeneration purposes. Unfortunately, these membranes degrade in the body over a period of time and may produce toxic degradation products and may cause adverse tissue reaction.

There are other medical applications where existing treatment methods are not completely satisfactory. For example, one of the principal reasons for the regenerative failure in the nerve system after injury, is the interface of normal neural tissue and glial scar tissue that forms after injury and prevents axon growth. Impaired neurological function resulting from injuries and diseases has been treated by biomaterial-based therapies. The inability of transplanted or regenerating neurons to grow over long distances through scar tissue has led to the development of bridging substrates that serve to direct axonal regeneration to appropriate biological targets. However, these synthetics materials have failed to provide optimal effects on neuronal attachment, and as scaffolding to optimize neurite outgrowth and directionality.

As another example, cartilage does not regenerate well after injury or once diseased. No natural or synthetic materials have been successfully utilized to promote the healing of cells at that location, such as meniscus, intervertebral disk.

In addition, various collagen-based or synthetic materials have been utilized for wound closure, such as for skin ulcer, burn, and after pneumonectomy, etc. however, these materials either did not exhibit the requisite tensile strength or also had problems with infection and other immune responses, encapsulation, or had other problems when they may have been loaded with antibiotics, growth factors and the like.

In another example, injectable crosslinked or non-crosslinked collagen compositions, as well as synthetic polymeric material, have been utilized to fill the space within soft tissue for the purposes of general or cosmetic surgery, such as to effect augmentation of facial or breast tissue. However, no ideal methods have been developed for treating these applications.

### SUMMARY OF THE DISCLOSURE

It is an object of the present invention to provide tissue material which does not suffer from the drawbacks associated with the graft materials identified above.

It is another object of the present invention to provide a method for preparing a tissue material from a section of a blood vessel.

It is yet another object of the present invention to provide a multi-purpose tissue material which can be used in autografting, allografting and heterografting applications.

It is yet a further object of the present invention to provide a tissue material that can be used to replace or repair damaged tissue structures.

It is yet another object of the present invention to provide a tissue composition which promotes endogenous tissue growth.

It is yet another object of the present invention to provide a tissue composition which can display various absorbablity (life span) in vivo.

It is yet another object of the present invention to provide a tissue material that can be used for wound closure.

It is yet another object of the present invention to provide a tissue material which can be used as a nerve regeneration guide for bridging.

It is yet another object of the present invention to provide a tissue composition that can be used in treating cartilage degeneration or injury, such as the meniscus and intervertebral disk.

It is yet another object of the present invention to provide a tissue material that can be used for ligament or tendon replacements, or as replacements for other connective tissues.

It is yet another object of the present invention to provide a tissue material that can be used in treating diaphyseal bone defects, and in bone regeneration applications.

It is yet another object of the present invention to provide a fluidized tissue material that can be injected into a mammalian host for general cosmetic surgeries.

In order to accomplish the objects of the present invention, there is provided a tissue composition having the subendothelial layer, the elastica interna, and at least a portion of the tunica media of a blood vessel harvested from a mammal. The tissue composition can also include a portion of the tunica adventitia of a blood vessel harvested from a mammal. In a preferred embodiment, the endothelial cells have been removed from the blood vessel.

The tissue composition of the present invention can be formed into a graft, a patch, a connective tissue for surgical repair, an orthopedic graft, and a substrate for cell growth, among other applications. The tissue composition of the present invention can also be fluidized, or made into powdered form.

The tissue composition of the present invention can be prepared by providing a blood vessel harvested from a mammal, the blood vessel having a subendothelial layer having endothelial cells provided thereon, a layer of elastica interna, a layer of tunica media, and a layer of tunica adventitia, then removing the endothelial cells, followed by removing a portion of the layer of tunica adventitia. It is also possible to remove the entire layer of tunica adventitia and a portion of the layer of tunica media. The base tissue material can then be chemically modified, such as by cross-linking or surface modification, to a different degree to effect different stabilities.

### BRIEF DESCRIPTION OF THE DRAWINGS

FIG. 1 is a cross sectional view of a section of a vascular blood vessel.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The term vascular “tissue” as used herein is intended to mean any mammalian (human or animal) tissue that is collected or harvested from the circulatory system of the mammal.

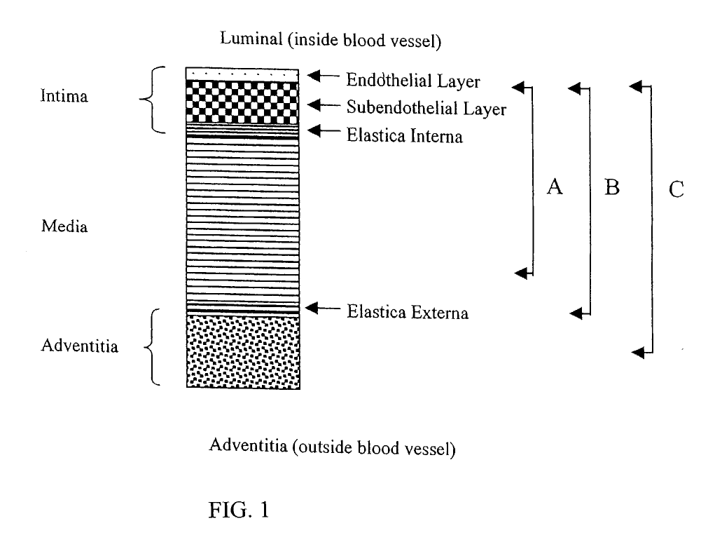

The present invention relates to a novel tissue composition and a method for preparation of the tissue composition from a segment of blood vessel. A possible tissue composition for the present invention can comprise the subendothelial layer, the elastica interna and a partial layer of the tunica media of a segment of blood vessel of a mammal. Alternatively, the tissue composition for the present invention can also comprise the subendothelial layer, the elastica interna, the layer of tunica media, and a partial layer of the tunica adventitia of a segment of blood vessel of a mammal. According to the present invention, the endothelial cells are removed from the tunica intima, and at least a portion of the tunica adventitia, and possibly a portion of the tunica media, are also removed. As a result, the resulting tissue composition of the present invention can comprise either the layers in A, B or C illustrated in FIG. 1. The endothelial cells are removed because the nature of heterogeneous and homogeneous endothelial cells can activate a strong immune response from the host or recipient. The outside portion of the tunica adventitia (which contains loose connective tissue and adipose tissue) is also removed entirely or partially to provide a uniform tissue composition. A portion of tunica media can also be removed to meet the thickness requirement for the desired application. As used herein, the term “outside surface” means the outermost layer of the tissue composition, which can either be the remaining partial layer of the tunica adventitia, or the remaining partial layer of the tunica media (if the tunica adventitia has been completely removed).

The basic organization of the wall of all arteries and veins is similar in that three concentric layers can be distinguished: (1) an inner layer, the tunica intima, which consists of an endothelial cell layer, a subendothelial layer and the elastica interna; (2) an intermediate layer, the tunica media, which is composed predominantly of smooth muscles; and (3) an outer coat, the tunica adventitia, made up of fibroblast and fibrous elements. The tunica adventitia gradually merges with the loose connective tissue that accompanies all blood vessels. The boundary between the tunica intima and tunica media is marked by internal elastic lamina (elastic interna), which is especially well developed in arteries of medium caliber. Between the tunica media and the tunica adventitia is a thinner external elastic lamina (elastica externa) that is also present in many arteries.

Arteries:

There is a continuous gradation in size and in character of the vessel wall from the largest arteries down to the capillaries, but it is customary to designate several categories of arteries on the basis of their size, the predominant structural component of their tunica media.

The large elastic or conducting arteries, such as the aorta, innominate, subclavian, common carotid, and common iliac, have walls containing many fenestrated layers of elastin in the tunica media. The tunica intima of these arteries consists of the endothelium, separated from the elastic interna by loose connective tissue containing a few fibroblasts, occasional smooth muscle cells, and sparse collagenous and elastic fibers. The elastica interna is a less prominent feature of elastic arteries than of muscular arteries, and is the first of many laminae of elastin found in the tunica media. The tunica media is up to 500 μm thick in the larger vessels and is composed of 40-70 fenestrated laminae of elastin 5-15 μm apart and interconnected by radically oriented finer strands of elastin. The spaces between elastic laminae are occupied by long branching smooth muscle cells, occasional fibroblasts, with associated collagen fibers and other extracellular matrix components. The elastic externa is relatively thin and inconspicuous, being simply the most peripheral of the multiple elastic laminae of the tunica media. The tunica adventitia of elastic arteries is relatively thin, and consists of fibroblasts and collagen bundles of elastic fibers.

Elastic arteries gradually give way to muscular or distributing arteries such as the brachial, femoral, radial, popliteal, and their branches. This category includes the majority of the vessels in the arterial system and spans a wide range of sizes down to half a millimeter in diameter. For these arteries, the tunica intima is somewhat thinner than that of the elastic arteries and lacks smooth muscle cells but is otherwise similar in its organization. Beneath the tunica intima is a well-developed elastica interna. The thickness of the tunica media varies from 40 layers of smooth muscle cells in larger arteries to three or four layers in small arteries. In the external lamina, the smooth muscle cells are embedded within abundant interstitial collagen fibrils and loose networks of thin elastic fibers. The elastica externa often appears to be a continuous lamina at the boundary between the tunica media and the tunica adventitia. The tunica adventitia of muscular arteries may be thicker than the tunica media and also consists of fibroblasts, elastic fibers, and bundles of collagen. The tunica adventitia continues into the surrounding connective tissue without a clearly defined boundary.

Veins:

It is customary to distinguish three types of veins: veins of small, medium and large caliber. The three layers (i.e., the tunica intima, the tunica media, and the tunica adventitia) of the vessel wall may be distinct. The major components of the veins are muscular and elastic tissue.

Veins of small caliber (0-2 mm) and capillaries can be difficult to harvest and do not always have good mechanical strength, so they may not be applicable to all applications described herein.

Veins of medium caliber (2-9 mm) include the cutaneous and deeper veins of the extremities distal to the brachial and the popliteal, and the veins of viscera and head, with the exception of the main trunks. In the tunica intima of these veins, there are endothelial cells and connective tissue layer with a few cells and thin elastic fibers. The tunica media is much thinner than in the arteries and consists mainly of circular smooth muscle fibers separated by many longitudinal collagenous fibers and a few fibroblasts. The tunica adventitia is usually much thicker than the tunica media and consists of loose connective tissue with collagenous bundles and elastic networks. The layers adjacent to the tunica media often contain a number of longitudinal smooth muscle bundles.

In the veins of large caliber, the tunica intima has the same structure as in the medium-sized veins. In some of the larger trunks, the connective tissue layer is of considerable thickness. The tunica media is sometimes absent from the structure of large caliber veins and large caliber trunks. The structure of large caliber veins and large caliber trunks can be the same as the structure for veins of medium caliber.

Preparation:

The vascular tissue composition of the present invention is prepared by removing the endothelial cells and at least a partial layer of the tunica media and/or the tunica adventitia. To remove endothelial cells, the luminal (inner) surface of the vascular tissue can be subjected to a solution with low osmolarity to burst the endothelial cells. The endothelial cells can also be removed by prolonging the storage time of the vascular tissue in a refrigerator until necrosis of the endothelial cells. Then, the remnant or debris of the endothelial cells can be removed physically by mild abrasion, repeatedly flushing the lumen of the vessel with saline, or by sonication, or chemically by enzymatic digestion. The tunica adventitia and tunica media (if desired to be removed) can be removed using many techniques, such as surgical dissection, and machine or laser cutting on a mandrel, among others. The finished vascular tissue composition contains mainly collagen, elastin and smooth muscle cell.

The prepared graft material is typically rinsed with alcohol to reduce the bio-burden (i.e., to kill residual bacterial), followed by chemical treatment, such as cross-linking or modification, that are well-known in the art. The vascular tissue composition can be sterilized using most well-known sterilization techniques, such as radiation, chemical sterilization (aldehyde based, iodine based sterilant), electron beam, UV, ETO, etc.

Depending on their locations and functions, the tissue composition of arteries and veins varies. The proper selection of vascular tissue can provide tissue with different elasticity, different strength and stretchability, and variable thickness (from 5 μm-5 mm) with the ability to be used as multiple layers as well. As explained in greater detail hereinbelow, the tissue composition can be used in the following forms: (1) its natural configuration, (2) dry powder, (3) suspension or solution, and (4) gel or foam. The injectable foam and gel-like materials can be easily made and applied.

The vascular tissue composition can also be remodeled in vivo, and may be slowly resorbed by the host within months in general. In other words, heterogeneous tissue will be slowly absorbed in the host without sufficient cross-linking. The integration time varies and can be designed into the material composition by varying the degree of cross-linking. One advantage of the vascular tissue composition of the present invention is that it behaves like a scaffold for host endogenous tissue to grow by providing an ideal substrate. Once the host tissue has grown back completely, the vascular composition is withdrawn based on the designed lifespan. As a result, the vascular tissue may be chemically crosslinked (e.g., using glutaraldehyde, polyepoxy, PEG, UV, etc.) or not (i.e., using fresh, frozen or, cryopreserved tissue) depending on the desired applications. However, the implantable materials can be crosslinked, so that the stability of the materials can be varied from weeks to a life time during in vivo application.

Applications:

The vascular tissue composition of the present invention can be used in a number of applications.

Grafting Material for Blood Vessels: The vascular tissue composition of the present invention can be used as a graft to replace or repair blood vessels, as illustrated in greater detail in Examples 1-3 hereinbelow.

As a surface for cell attachment and growth: Research has been done to apply natural protein, such as collagen, to synthetic surfaces to promote or improve cell attachment and growth on synthetic plastic surfaces. In this regard, it is advantageous to the present invention that natural vascular tissue from mammals mostly contains smooth muscle, collagen fiber, and elastin, which provide a natural and ideal surface and matrix composition for cell growth both in vitro and in vivo.

The cell growth has been shown to be highly related to the surface chemical composition and topographic characteristics. The natural composition and configuration of vascular tissue provides a unique cell growth substrate that promotes the attachment and proliferation of cells. It is reasonable to believe that the luminal surface of the vascular composition promotes the growth of endothelial cells and the outside surface will promote the growth of a thin layer of the loose connective tissue. The vascular tissue substrates of the present invention provide cells with a collagenous matrix environment in vitro that resemble those found in vivo.

The present invention can provide mammalian vascular tissue-derived matrices as substrates for the growth and attachment of a wide variety of cell types. The collagenous matrices for use in accordance with the present invention comprise highly conserved collagens, glycoproteins, proteoglycans, and glycosaminoglycans in their natural configuration and natural concentration. Mammalian vascular tissue is a plentiful by-product of commercial meat production operations and is thus a low cost cell growth substrate, especially when the vascular tissue is used in its native layer tube/sheet configuration.

As a general material for surgical use: The present invention provides novel tissue graft constructs that can be used to promote regrowth and healing of damaged or diseased tissue structures, such as bone, ligament, tendon, cartilage and muscle, among others. More particularly, the vascular tissue composition of the present invention can be used as connective tissue substitutes, and in the following surgical applications: surgical repair of ligaments and tendons, as a surgically applied bone wrap to promote healing of bone fractures, as a tissue patch applied to abdominal defects or used with urinary bladder defects, and as a dermal graft applied to burn surfaces and non-healing skin ulcer.

It has been found that the grafts formed and used in accordance with the tissue composition of the present invention undergo biological remodeling upon implantation. Each of these grafts serves as a rapidly vascularized matrix for the support and growth of new endogenous connective tissue. The graft material of the present invention has been found to be trophic for host tissues with which it is attached or otherwise associated in its implanted environment. In multiple experiments, the graft material without cross-linking treatment has been found to be remodeled (resorbed and replaced with autogenous differentiated tissue) to assume the characterizing features of the tissue(s) with which it is associated at the site of implantation.

In one possible application, graft constructs of the present invention can be used as connective tissue substitutes, particularly as a substitute for ligaments and tendons. The graft can be formed from a segment of vascular tissue of a mammal. The graft can be used in its natural tubular shape or cut open and mounted on a frame (with or without stretch) to form a sheet. For tendon and ligament replacement applications, the resulting segment is typically preconditioned by stretching in both dimensions. The graft segment can then be formed in a variety of shapes and configurations, for example, to serve as a ligament or tendon replacement, or a substitute or a patch, for a broken or severed tendon or ligament.

In a ligament replacement application, opposite ends of the graft are attached to first and second bones, with the bones typically being articulated as in the case of a knee joint. It is understood that ligaments serve as connecting tissue for bones (i.e., between articulated bones), while tendons serve as connecting tissue to attach muscle to a bone. For example, one of the end portions of the graft may be adapted to be pulled through a tunnel in, for example, the femur and attached thereto, while the other of the end portions may be adapted to be pulled through a tunnel in the tibia and attached thereto to provide a substitute for the natural cruciate ligament. The graft would be adapted to be placed under tension between the tunnels to provide a ligament function (i.e., a tensioning and positioning function) that is provided by a normal ligament. Vascular tissues made of arteries have been found to have good mechanical strength characteristics for use as a tendon or ligament graft.

In tendon and ligament replacement studies, the graft appears to develop a surface that is synovialized. Additionally, the boundaries between the graft and endogenous tissue are no longer discernible. Indeed, where a single graft “sees” multiple micro-environments when implanted, it is differentially remodeled along its length. Thus, when used in cruciate ligament replacement experiments, not only does the portion of the graft traversing the joint become vascularized and actually grow to look and function like the original ligament, but the portion of the graft in the femoral and tibia bone tunnels rapidly incorporates into and promotes development of the cortical and cancellous bone in those tunnels. In fact, it has been found that, after six months, it is not possible to identify the tunnels radiographically. As a result, it appears that vascular tissue serves as a matrix for and stimulates bone regrowth (remodeling) within the tunnels.

In another possible application, the tissue composition of the present invention can be used to provide an orthopaedic graft or membrane for use as connective tissue to hold fractured bone pieces together and in proper orientation in the body, with the graft or membrane being formed to serve as a fracture wrap about segments of fractured bone and to be attached to the bone.

In another embodiment, the membrane can be shaped in a tubular form, preferably with a longitudinal slit to allow variability of the tubular diameter over a wide range. The diameter of the tubular membrane should match the dimensions of the bone to be treated, for example, 4.0 to 50.0 mm for tibia reconstruction. A thin membrane (e.g., 0.5 mm thickness) can also be rolled up either pre- or intraoperatively to form a final tubular implant (e.g., with a wall thickness of about 3.0 mm).

In one embodiment, the overlapping and intersecting tubular membrane provides semiocclusive fits at the two extremities of the tube for better external adaptation to the cortical bone ends and/or internal adaptation within medullary canals of long or tubular bones. The internal membrane tube to be inserted into an intramedullary canal should be formed on a mandrel which has an external diameter that is at least 1.1 to 3.0 times larger than the diameter of the intramedullary canal, and the external membrane tube should be formed on a mandrel which has an external diameter which is at least 1.1 to 3.0 times smaller than the external diameter of the bone. Due to its elasticity, the tubular membrane can be easily placed and fitted on the bone to be treated. If two tubular membranes are used (an external membrane over the cortical bone ends and an internal membrane within the medullary canal), they can either be used with or without spacers between the two tubular membranes. The two membranes can either be supplied pre-operatively as a single unit (with interconnecting structures), or can be assembled intra-operatively by the surgeon. Accordingly, one membrane is rolled in a tubular fashion to obtain a press fit into the medullary canal of both bone fragments, and a second membrane is rolled into a tube to form a semiocclusive external sleeve, bridging the two bone fragment ends. The space between the two membranes forms a new artificial cortex that can be filled with a variety of therapeutic substances, such as autologous bone, allogenic bone, demineralized bone powder extracts, bone morphogenetic protein isolates, antibiotics, or antineoplastic drugs. The walls of the membranes can either be continuous or intersected longitudinally for better adaptation of the membrane on the bone. If necessary, the space between the inner and outer tubular membranes can be maintained in their relative positions by means of spokes, ribs or other corrugated elements attached permanently, or inserted between, the membrane tubes upon surgery.

The membrane tube can be used as a complete tubular construction or only as a part of the construction (e.g. a partial tube) depending on the bone defect to be treated. The membranes according to the present invention can be used for a variety of applications to span osseous defects that have been conventionally stabilized with an external fixator, intramedullary rod, or systems of plates and screws, of which the most important are listed below:

(1) as a tissue separator which promotes and protects osseous regeneration, for the treatment of osseous defects secondary to trauma, infection, neoplasm or surgical resection;

(2) as a cortical spacer across bone defects;

(3) as an osseous activity barrier that continues bone regeneration to desired regions and prevents the formation of synostosis, physeal bars, or ectopic bone;

(4) as a spacer for skeletal resection, revision arthroplasty, or arthrodesis of human joints;

(5) as a container for autologous or allogenic graft materials: and

(6) as a combined bone grafting and/or drug delivery system.

As a result, the membrane according to the present invention offers a novel treatment that reliably promotes osseous healing, with and without adjunctive therapies, while posing no toxic risks or requirements for extensive surgery for implantation or removal. The membrane can be pre-operatively shaped and adjusted by the surgeon to conform and integrate with a wide range of anatomical defects and locations. Thus, the advantages offered by this membrane of the present invention include:

(1) maintenance of a physical barrier that contains osseous activity of the host bone, while simultaneously protecting this osseous activity from nonosteogenic and interfering cell lines; and

(2) protection of the cortical and medullary canal osteogenic capacity from surrounding soft tissues and inflammatory reactions.

In yet another possible application, the vascular tissue composition of the present invention can be used as a bridging substrate for nerve regeneration. One of the principal reasons for regenerative failure in the nerve system after injury is the interface of normal neural tissue and glial scar tissue that forms after injury and prevents axon growth. Impaired neurological function resulting from injuries and diseases has been treated by biomaterial-based therapies. The inability of transplanted or regenerating neurons to grow over long distances through scar tissue has led to the development of bridging substrates that serve to direct axonal regeneration to appropriate biological targets. However, these methods have failed in providing optimal effects on neuronal attachment, and as scaffolding to optimize neurite outgrowth and directionality.

In yet another possible application, the vascular tissue composition of the present invention can be used as a tissue graft for replacing/reconstructing damaged or diseased urothelial tissue of the urinary tract. Urothelial tissue is defined herein as including the cells and tissues of the ureter, urethra and renal collection system. The vascular tissue graft constructs of the present invention promote the growth of endogenous urothelial tissues upon implantation into a host organism, and can be used to replace large sections of the ureter, urethra and urinary bladder.

In one embodiment, the vascular tissue graft constructs are surgically implanted to promote the growth of endogenous urinary bladder tissues having a urine-impermeable cell layer and a functional muscle cell layer. The tissue graft material serves as a scaffold and induces the proliferation of functional urinary bladder tissue formation which replaces the tissue graft without shrinkage of the graft area or formation of “scar” tissue. As mentioned herein, the tissue graft construct can be formed in the shape of a pouch to assist in the replacement of large portions (i.e. greater than 20%) of the urinary bladder. The tissue graft construct can be formed by surgical construction from one or more vascular tissue strips. Furthermore, the tissue graft construct can be manipulated to form ports in the tissue graft for surgical attachment and fluid communication with the ureters and the urethra.

The tissue graft constructs of the present invention can also be implanted into a vertebrate host species to repair a damaged, diseased or otherwise functionally compromised urinary bladder. In one embodiment, the defective portion of the bladder is surgically removed and replaced with a tissue graft construct of the present invention. The luminal side of the vascular tissue is preferably directed toward the bladder lumen. Large portions of the bladder can be removed and replaced with the tissue grafts of the present invention. After implantation, the constructs are eventually remodeled by the host with functional urinary bladder tissues having a stratification of cell layers similar to that found in the normal urinary bladder wall. It is anticipated that the entire urinary bladder can even be replaced with the present graft construct to regenerate a functional urinary bladder.

In yet another possible application, the vascular tissue composition of the present invention can be used as a method to deliver any drugs locally, and in particular, growth factors to induce cells responsible for the repair, healing and augmentation of tissues and organs. Drug can be absorbed into vascular tissue or encapsulated by multiple layers of the vascular tissue configuration. Applied growth factor element-containing compounds are suitable for a variety of applications such as wound healing, bone fracture repair, treatment of dermatologic conditions and successful improvement of bio and hemocompatibility of implanted tissue grafts and prostheses.

The vascular tissue can be fluidized: The present invention provides an injectable tissue graft composition and methods for its preparation and use. More particularly, the present invention provides injectable, non-immunogenic tissue graft compositions derived from vascular tissue. Upon deposition in vivo in an area of a tissue defect, the tissue graft compositions promote growth of endogenous tissue to repair the defect.

The vascular tissue composition described hereinabove can be fluidized by comminuting and/or protease digestion, without loss of its apparent biotropic properties, for use in less invasive methods of administration (e.g., injection or topical) to host tissues in need of repair. The fluidized composition is used advantageously in a method for inducing formation of endogenous tissue including bone and soft tissues such as muscle and connective tissues in mammals. The method comprises the step of injecting into the mammal a composition comprising a fluid or suspension of comminuted specially prepared vascular tissue or a protease digest thereof in an amount effective to induce endogenous tissue growth in the locale of the injected fluidized tissue graft composition. Endogenous connective tissues induced to grow in accordance with the present invention include collagen, elastin and muscle.

As another example, a method is provided for augmenting sphincter function in a mammal, the method comprising the step of injecting into tissue forming the sphincter an effective amount of a tissue graft composition comprising an aqueous suspension of comminuted specially prepared vascular tissue. As an alternative, the injectable composition can comprise protease digested specially prepared vascular tissue.

The tissue compositions of the present invention can be used for surgical reconstruction of a collagenous meniscus at the interface of articulating bones. In such reconstruction, a sheet of a first tissue graft composition, preferably itself comprising the vascular tissue of the present invention, is formed into a sealed pouch and filled with a fluidized tissue graft composition according to the present invention.

A similar approach can be used for breast reconstruction. The fluidized vascular tissue composition (liquid, gel or sponge form) can be injected or implanted into or underneath breast tissue to promote growth of endogenous tissue. A sealed pouch made of vascular composition, filled with a fluidized tissue composition can also be implanted into or underneath the breast tissue.

The present invention can also provide a cell culture composition including a proliferating cell population in contact with vascular tissue of a mammal and a nutrient medium for support of the growth of special cell population, in vitro as illustrated in Example 9 hereinbelow. Thus, the tissue composition of the present invention can be used as a growing substrate (e.g., like a petrie dish) to study cell behavior.

As an example of an application for cell culture growth, the present invention can provide a model system for studying tumor cell growth. The model system can comprise a proliferating tumor cell population in contact with a nutrient medium and vascular tissue composition according to the present invention to study the behavior of tumor cells.

As yet a further example, the present invention can enhance or expand the functional properties of mammalian vascular tissues as an implantable or injectable tissue graft construct by first seeding the vascular tissue in vitro with preselected or predetermined cell types (e.g. autogenous endothelial cells) prior to implanting or injecting the graft construct into the host. In other words, there are some applications (e.g., using small caliber veins) where it is beneficial to first sed endothelial cells of a host or recipient onto the tissue composition before implantation into the recipient.

The fluidized vascular tissue of this invention also finds use as an injectable heterograft or homograft for tissues, for example, bone or soft tissues, in need of repair or augmentation, but most typically to correct trauma or disease-induced tissue defects. The fluidized vascular tissue compositions of the present invention are also used advantageously as a filler, with one or more sheets of the tissue formed into sealed pouches or “pillows” for use in cosmetic or trauma-treating surgical procedures.

The fluidized tissue compositions of the present invention can be prepared as solutions or suspensions of vascular tissue by comminuting and/or digesting the vascular tissue with a protease, such as trypsin or pepsin, for a period of time sufficient to solubilize said tissue and form a substantially homogeneous solution. The vascular starting material is comminuted by tearing, cutting, grinding, shearing and the like. Grinding the tissue in a frozen or freeze-dried state is preferred although good results can be obtained as well by subjecting a suspension of pieces of the tissue to treatment in a high speed blender and dewatering, if necessary, by centrifuging and decanting excess water. The comminuted vascular tissue can be dried to form a tissue powder (as described below). Thereafter, it can be hydrated, that is, combined with water or buffered solution and optionally other pharmaceutically acceptable excipients to form a tissue graft composition as a fluid having a viscosity of about 2 to about 300,000 cps at 25° C. The higher viscosity graft compositions can have a gel or paste consistency. The tissue compositions of the present invention can be sterilized using well-known sterilization techniques such as exposure to ionizing radiation or electron beam.

The Vascular Tissue can be in Powdered Form: Vascular tissue powder can be prepared in the following manner. Vascular tissue specimens prepared as described above are minced or chopped into small pieces using tissue scissors, a single-edged razor blade, or other appropriate cutting implement. The specimens are placed in a flat bottom stainless steel container and liquid nitrogen is introduced into the container to freeze the specimens to prepare them for comminuting. The frozen vascular tissue specimens are then comminuted to form a coarse vascular tissue powder. Such processing can be carried out, for example, with a manual arbor press with a cylindrical brass ingot placed on top of the frozen specimens. Other methods for comminuting vascular tissue specimens may be utilized to produce a vascular tissue powder usable in accordance with the present invention. For example, vascular tissue specimens can be freeze-dried and then ground using a manual arbor press or other grinding means. Alternatively, vascular tissue can be processed in a high shear blender to produce, upon dewatering and drying, a vascular tissue powder. Again, liquid nitrogen can be used as needed to maintain solid frozen particles during final grinding. The powder can be easily hydrated using, for example, buffered saline to produce a fluidized tissue graft material of this invention at the desired viscosity.

Thus, the vascular tissue composition according to the present invention has many advantages over the other known materials. For example, when compared with SIS, the tissue compositions of the present invention have higher elasticity, are free from residual bacteria and partially digested food proteins and lipid, have high purity, have variable amounts of SMC and collagen or elastin content, and have low lipid content. The vascular tissue composition of the present invention is also an ideal surface for supporting endothelial cell growth (low thrombogenicity), and provides high strength and variable absorbability.

### EXAMPLE 1

### Porcine Artery as a Venous Graft

In this study, a porcine thoracic aorta that has been cross-linked with epoxy had the entire tunica adventitia and endothelial cells removed. This porcine graft was studied in an extreme low blood flow environment in 23 beagle dogs as inferior vena Cava (IVC) replacement. The graft averaged 9.0 mm in diameter and 5 cm in length. Without systematic anticoagulation, the porcine graft had a 96% first week patency and a 70% six month patency. They are superior to the patency from a control group of Gortex ePTFE in 9 dogs, which had a patency of 56% at two days after surgery and 44% at 6 months post-operation.

At three months post-operation, the porcine graft had been completely endothelialized and well-accepted by the host. No adverse tissue reactions (lymphocyte and macrophage infiltration) were observed at the adventitial side and at the anastomotic regions. At retrieval, the porcine graft retained the original physical property—natural blood vessel compliance. The host fibrous encapsulation was minimal for the porcine grafts, while the Gortex ePTFE graft had a very thick fibrous tissue which completely covered the outside of the graft and extended 2 cm proximal and distal of the anastomosis on to the host IVC. The Gortex graft had become rigid.

The porcine subendothelial layer becomes a scaffold for host endothelial cells to populate on its natural surface. The outside layer accommodated the host connective tissue very well. A minimal amount of loose connective tissue attached to the graft.

### EXAMPLE 2

### Porcine Artery as Esophageal Graft

In this study, a composite porcine thoracic aorta was formed by suturing two aorta together laterally at 2 cm in diameter, with epoxy fixation treatments. The composite porcine thoracic aorta had its entire tunica adventitia and endothelial cells removed, and was used as esophageal replacement in 10 mongrel dogs. The graft averaged 7 cm in length. 0% mortality was achieved. Immediately after implantation, a fibrous encapsulation was noted around the graft, which prevents the leakage of air, food, and bacterial from the esophagus into the chest cavity. By two weeks post-procedure, the fibrous tissue on the graft surface had been gradually replaced by host smooth muscle cells. By two months post-procedure, regrowth of the entire smooth muscle conduit was completed. Then, the porcine graft was separated from the muscle conduit and dropped into the stomach. At the end of three months, host epithelial cells had migrated and completed the coverage on the muscle conduit of the host.

The porcine tunica media becomes a scaffold first for mesenchymal cells attachment with high success rate in early post-surgical survival, and later for host smooth muscle cells to populate on its natural surface.

### EXAMPLE 3

### Porcine Vein as Stent Covering Graft

In a first study, porcine vein, harvested from the extremities and neck region, was cross-linked with epoxy. The endothelial cells were removed before chemical cross-linking. All the tunica adventitia and part of the tunica media were carefully removed from the blood vessel using surgical dissecting technique after the completion of the fixation process. The vein was slid on to the outside of a coronary stent (4.0 mm in diameter and 25 mm in length) and then sutured to its proximal and distal edges. A total of 10 vein-covered stents were implanted in iliac arteries of 10 mongrel dogs. At 6 months all stents with porcine cover were patent. The explant showed no signs of thrombus on the luminal surface of the covered stent.

In a second study, the covering materials were porcine abdominal vein having undergone the same treatment as above. A total of 10 porcine vein-covered stents were evaluated in 10 animals using a nondiseased swine coronary artery model to determine the vascular response to stent implantation.

In the ten animals who received coronary stents, one stent had incomplete deployment. The result from that animal was excluded. Nine stents were patent with mean observation time of 141 days. The excellent hemocompatibility and deliverability of the stent cover are demonstrated in this standard porcine coronary arterial model. The subendothelial layer of the porcine vein was used for supporting host endothelial cell growth in this application.

### EXAMPLE 4

### Vascular Tissue for Local Drug Delivery

The coronary stent cover (in Example 3) can be made of double layers of porcine venous tissue. Functional drug or other reagents can be encapsulated within the chamber formed by the two layers of tissue. The vascular tissue behaved like a diffusion membrane which allows drug to be released slowly within a period of time after implantation.

In one study, Taxol (Taxol 0.5 mg) was loaded between two layers of porcine tissue on a standard coronary stent. The covered stent with drug was delivered into a porcine coronary artery. The drug was gradually released to the diseased artery and further inhibited the growth of smooth muscle cells.

### EXAMPLE 5

### Vascular Tissue as Bone Regeneration Composition

Two different bone graft compositions comprising vascular tissue according to the present invention were investigated for their induction of endogenous growth of bone tissue. One formulation consisted of vascular tissue in its natural hydrated state and the other represented vascular tissue as a lyophilized particulate.

Prior to implantation, the material was minced into pieces no larger than 1×1 mm² and rinsed in sterile saline. Vascular tissue as a dehydrated particulate was prepared by pulverizing the tissue under liquid nitrogen to achieve particles which ranged from 0.1 to 1 mm² in size. The particulate was then lyophilized overnight and then sterilized to form a solid anhydrous particulate composite.

Lyophilized particulate compositions of the present invention can be molded, pressed, and contoured, for example, to fit a bone defect. In addition, composites involving vascular tissue and various other materials (e.g., hydroxyapatite, tricalcium phosphate, etc.) may be formed and useful for tissue repair. The lyophilized vascular tissue compositions of the present invention find wide application both in bone tissue replacement and repair. These studies showed that bone graft compositions used in accordance with the present disclosed method can induce regrowth of natural connective tissue or bone in an area of an existent defect.

Damaged or defective regions of bone can be repaired by contacting the damaged or defective region with a bone graft composition comprising vascular tissue. Perhaps the most remarkable aspect of the tissue compositions of the present invention is their ability to induce regrowth of natural bone tissue in an affected area. These studies showed that, by contacting a damaged or defective region of bony tissue with an effective amount of the present lyophilized vascular tissue composition, one can induce natural bone growth and repair of the damaged or defective region. Additional bioactive agent, e.g., physiological compatible minerals, growth factors, antibiotics, chemotherapeutic agents, antigens, antibodies, enzymes and hormones, can also be mixed with the tissue composition and bone graft to promote the regeneration of endogenous tissues.

### EXAMPLE 6

### Fluidized Vascular Tissue as An Injectable Tissue Graft

Vascular tissue powder according to the present invention is subjected to proteolytic digestion to form a substantially homogeneous solution. In this study, the powder was digested with 1 mg/ml of pepsin in 0.1 M acetic acid, adjusted to pH 2.5 with HCl, over a 48 hour period at room temperature. The reaction medium was neutralized with sodium hydroxide to inactivate the peptic activity. The solubilized vascular tissue may then be concentrated by salt precipitation of the solution and separated for further purification and/or freeze drying to form a protease solubilized vascular tissue in powder form.

The viscosity of fluidized vascular tissue compositions in accordance with the present invention can be manipulated by controlling the concentration of the vascular tissue component and the degree of hydration. The viscosity can be adjusted to a range of about 2 to about 300,000 cps at 25° C. Low viscosity vascular tissue compositions are better adapted for intraarticular applications or applications within body cavities. Higher viscosity formulations, for example, gels, can be prepared from the Vascular tissue digest solutions by adjusting the pH of such solutions to about 6.0 to about 7.0. Gel forms of the present compositions, as vascular tissue or vascular tissue digest solutions, are typically preferred for subcutaneous or intramuscular applications using syringes or catheters.

Perhaps the most remarkable aspect of the tissue compositions of the present invention is their ability to induce regrowth of natural tissue in an affected area, as shown in this study. By injecting an effective amount of a fluidized vascular tissue composition into the locale of a tissue defect or a wound in need of healing, one can readily take advantage of this surprising property without the need for major invasive operations.

### EXAMPLE 7

### Fluidized, Injectable Vascular Tissue Composition for Meniscus Repair

Vascular tissue as a suspension may be utilized as a meniscus. This vascular tissue meniscus consists of a sheet of vascular tissue according to the present invention (with the luminal surface forming an “outside” layer or surface) formed into a semicircular pillow. The pillow was then filled with a suspension of vascular tissue and the suture line of the pillow was attached to the medial collateral ligament. Thus, the substance of the pillow served as a weight-bearing shock absorber between the medial femoral condyle and the tibial plateau. The same approach can be used to treat disorders of the intervertebral disk.

The fluidized vascular tissue compositions of this invention find wide application both in tissue replacement and repair. The fluidized vascular tissue compositions are used in accordance with the present method to induce regrowth of natural connective tissue or bone in an area of an existent defect, as shown in this study. By injecting an effective amount of a fluidized vascular tissue composition into the locale of a tissue defect or a wound in need of healing, one can readily take advantage of the biotropic properties of the tissue composition without the need for more invasive surgical techniques.

### EXAMPLE 8

### Vascular Tissue for Breast Tissue Engineering

An approach similar to those in Example 7 can be used for breast reconstruction. The fluidized vascular tissue composition (liquid, gel or sponge form) can be injected or implanted into or underneath breast tissue to promote growth of endogenous tissue.

A sealed pouch made of vascular composition that is filled with a fluidized tissue composition can also be implanted into or underneath breast tissue.

### EXAMPLE 9

### Vascular Tissue for in vitro Cell and Tissue Culture

Vascular tissue according to the present invention was prepared and sterilized via various techniques (gamma irradiation, peracetic acid, etc.), and the vascular tissue was clamped within a polypropylene frame to create a flat surface area (50 mm²) for cell growth to study cell behavior. The frame was submerged in culture medium to allow access of medium nutrients to both surfaces of the vascular tissue. Various cell types were seeded on the vascular tissue and then placed in a 5% CO₂, 95% air incubator at 37° C. Various cell growth characteristics were determined.

The cellular growth on both the luminal and adventitia sides of the vascular tissue was investigated. Vascular tissue as a growth substrate exhibits sidedness; that is, the cell/matrix interactions are different when the cells are cultured on the adventitia versus the luminal side of the vascular tissue. When selected cell types, such as rat FR cells, are seeded on the luminal side, the cells attach to the matrix surface and proliferate to form a cellular polylayer. Alternatively, when FR cells are seeded on the adventitia side, the cells not only grow along the surface but also migrate into the vascular matrix.

The luminal side of mammalian vascular tissue provides a dense connective tissue matrix and more readily supports monolayer or polylayer formation of select cell types (i.e. endothelial and epithelial cells). Alternatively, the adventitia side represents a more loose connective tissue structure that more readily supports migration of cells within the matrix structure (i.e. fibroblasts).

Fluidized vascular tissue can also be used in cell culture media, as a nutrient for promoting cell growth.

While the description above refers to particular embodiments of the present invention, it will be understood that many modifications may be made without departing from the spirit thereof. The accompanying claims are intended to cover all such modifications as would fall within the true scope and spirit of the present invention.

What is claimed is:

1. A tissue composition comprising the subendothelial layer, the elastica interna, and at least a portion of the tunica media of a blood vessel harvested from a mammal.

2. The composition of claim 1, further comprising a portion of the tunica adventitia of a blood vessel harvested from a mammal.

3. The composition of claim 1, wherein the endothelial cells have been removed from the blood vessel.

4. The composition of claim 1 formed into a graft.

5. The composition of claim 1 formed into a patch.

6. The composition of claim 1 formed into a connective tissue.

7. The composition of claim 6, wherein the connective tissue functions as a ligament, tendon, bone, cartilage, fixation structure for internal organs, body wall, or urothelial tissue.

8. The composition of claim 1, formed into a scaffold for repair or enhancement of other types of tissue.

9. The composition of claim 6, wherein the composition functions as epithelial, endothelial, nerveor muscular tissue.

10. The composition of claim 1 formed into an orthopedic graft.

11. The composition of claim 1 formed into a cell culture proliferation composition.

12. The composition of claim 1 formed into a substrate for cell growth.

13. The composition of claim 1 formed into a fluidized tissue composition and injected into a host.

14. The composition of claim 1 formed into a powdered tissue composition.

15. The composition of claim 4, further including a drug or growth factor incorporated with the graft.

16. The composition of claim 1, wherein the composition is chemically modified or cross-linked to have different biostabities.

17. A method of preparing a tissue composition for medical use, comprising:
providing a blood vessel harvested from a mammal, the blood vessel having a subendothelial layer having endothelial cells provided thereon, a layer of elastica interna, a layer of tunica media, and a layer of tunica adventitia;
removing the endothelial cells; and
removing a portion of the layer of tunica adventitia.

18. The method of claim 17, further including:
removing the layer of tunica adventitia; and
removing a portion of the layer of tunica media.

19. The method of claim 17, further including:
chemically modifying the composition.

20. A method of surgically repairing diseased or damaged tissue structures comprising a first tissue structure and a second tissue structure, comprising:
providing a graft construct made from a tissue composition comprising the subendothelial layer, the elastica interna, and at least a portion of the tunica media of a blood vessel harvested from a mammal, the graft construct having a first end and a second end;
attaching the first end of the graft construct to the first tissue structure; and
attaching the second end of the graft construct to the second tissue structure.

21. The method of claim 20, wherein the tissue composition further comprises a portion of the tunica adventitia of a blood vessel harvested from a mammal.

22. The method of claim 20, wherein the first and second tissue structures are selected from the group consisting of bone, ligament, tendon, cartilage and muscle.

23. A method of forming a fluidized tissue composition, comprising:
providing a blood vessel harvested from a mammal, the blood vessel having the subendothelial layer, the elastica interna, and at least a portion of the tunica media, with the endothelial cells and a portion of the layer of tunica adventitia being removed; and
forming a protease digest of the blood vessel.

24. A method of forming a fluidized tissue composition, comprising:
providing a blood vessel harvested from a mammal, the blood vessel having the subendothelial layer, the elastica interna, and at least a portion of the tunica media, with the endothelial cells and a portion of the layer of tunica adventitia being removed; and
comminuting the blood vessel.

25. A method of inducing formation of endogenous tissue in a host mammal, comprising:
providing a fluidized tissue composition comprising a suspension of a blood vessel harvested from a mammal, the blood vessel having the subendothelial layer, the elastica interna, and at least a portion of the tunica media, with the endothelial cells and a portion of the layer of tunica adventitia being removed; and
injecting the fluidized tissue composition into the host mammal.

26. A tendon comprising a vascular tissue composition that comprises the subendothelial layer, the elastica interna, and at least a portion of the tunica media of a blood vessel harvested from a mammal.

27. A cartilage comprising a vascular tissue composition that comprises the subendothelial layer, the elastica interna, and at least a portion of the tunica media of a blood vessel harvested from a mammal.

28. A ligament comprising a vascular tissue composition that comprises the subendothelial layer, the elastica interna, and at least a portion of the tunica media of a blood vessel harvested from a mammal.
